(19) United States
(12) Patent Application Publication
(10) Pub. No.: US 2013/0172957 A1
(43) Pub. Date: Jul. 4, 2013

(54) NON-INVASIVE NERVE STIMULATOR CIRCUIT

(71) Applicants: Steven Mendez, New York, NY (US); Hechung Hu, Cedar Knolls, NJ (US); Arthur Ross, Mendham, NJ (US); Puyon Guo, Summit, NJ (US)

(72) Inventors: Steven Mendez, New York, NY (US); Hechung Hu, Cedar Knolls, NJ (US); Arthur Ross, Mendham, NJ (US); Puyon Guo, Summit, NJ (US)

(73) Assignee: ElectroCore, LLC., Morris Plains, NJ (US)

(21) Appl. No.: 13/715,256

(22) Filed: Dec. 14, 2012

Related U.S. Application Data
(60) Provisional application No. 61/580,756, filed on Dec. 28, 2011.

(51) Int. Cl. A61N 1/36

(52) U.S. Cl. CPC A61N 1/36014
USPC 607/59; 607/61

(57) ABSTRACT

The present disclosure describes a circuit that may be used in a non-invasive nerve stimulator. The circuit contains a signal source, a power source, an amplification source and a user interface portion. The circuit may be used in a handheld device that is applied directly to the surface of a patient or it may be used in other configurations.

### CROSS REFERENCE TO RELATED APPLICATIONS

This application claims the benefit of priority of U.S. Provisional Patent Application No. 61/580,756 filed Dec. 28, 2011. This application is also related to commonly assigned U.S. patent application Ser. No. 13/603,781 filed Sep. 15, 2012 which is a continuation-in-part of U.S. patent application Ser. No. 13/222,087 filed Aug. 31, 2011, which is a continuation-in-part of U.S. patent application Ser. No. 13/183,765 filed Jul. 15, 2011 which claims the benefit of priority of U.S. Provisional Patent Application No. 61/488,208 filed May 20, 2011 and is a continuation-in-part to U.S. patent application Ser. No. 13/183,721 filed Jul. 15, 2011, which claims the benefit of priority of U.S. Provisional Patent Application No. 61/487,439 filed May 18, 2011 and is a continuation-in-part of U.S. patent application Ser. No. 13/109,250 filed May 17, 2011, which claims the benefit of priority of U.S. Provisional Patent Application No. 61/471,405 filed Apr. 4, 2011 and is a continuation-in-part of U.S. patent application Ser. No. 13/075,746 filed Mar. 30, 2011, which claims the benefit of priority of U.S. provisional patent application 61/451,259 filed Mar. 10, 2011 and is a continuation-in-part of U.S. patent application Ser. No. 13/005,005 filed Jan. 12, 2011, which is a continuation-in-part of U.S. patent application Ser. No. 12/964,050 filed Dec. 9, 2010, which claims the benefit of priority of U.S. Provisional Patent Application No. 61/415,469 filed Nov. 19, 2010 and is a continuation-in-part of U.S. patent application Ser. No. 12/859,568 filed Aug. 9, 2010, which is a continuation-in-part of U.S. patent application Ser. No. 12/408,131 filed Mar. 20, 2009 and a continuation-in-part application of U.S. patent application Ser. No. 12/612,177 filed Nov. 9, 2009 the entire disclosures of which are hereby incorporated by reference.

### BACKGROUND

1. Technical Field

The following disclosure relates to a circuit for providing a non-invasive nerve stimulation signal.

2. Discussion of Technical Background

The use of electrical stimulation for treatment of medical conditions has been well known in the art for nearly two thousand years. It has been recognized that electrical stimulation of the brain and/or the peripheral nervous system and/or direct stimulation of the malfunctioning tissue, which stimulation is generally a wholly reversible and non-destructive treatment, holds significant promise for the treatment of many ailments.

Potential advantages of such non-invasive medical methods and devices relative to comparable invasive procedures are as follows. The patient may be more psychologically prepared to experience a procedure that is non-invasive and may therefore be more cooperative, resulting in a better outcome. Non-invasive procedures may avoid damage of biological tissues, such as that due to bleeding, infection, skin or internal organ injury, blood vessel injury, and vein or lung blood clotting. Non-invasive procedures are sometimes painless or only minimally painful and may be performed without the need for even local anesthesia. Less training may be required for use of non-invasive procedures by medical professionals. In view of the reduced risk ordinarily associated with non-invasive procedures, some such procedures may be suitable for use by the patient or family members at home or by first-responders at home or at a workplace, and the cost of non-invasive procedures may be reduced relative to comparable invasive procedures.

For example, transcutaneous electrical nerve stimulation (TENS) is non-invasive because it involves attaching electrodes to the surface of the skin (or using a form-fitting conductive garment) without breaking the skin. In contrast, percutaneous electrical stimulation of a nerve is minimally invasive because it involves the introduction of an electrode under the skin, via needle-puncture of the skin. Both TENS and percutaneous electrical stimulation can be to some extent unpleasant or painful, in the experience of patients that undergo such procedures. In the case of TENS, as the depth of penetration of the stimulus under the skin is increased, any pain will generally begin or increase.

Accordingly, a need exists for a circuit that can generate a painless, non-invasive signal, of the correct amplitude and frequency, to a nerve in a patient.

### SUMMARY

In an embodiment of the present disclosure the circuit is for a non-invasive nerve stimulator and comprises a signal generator having an output for generating a pattern of stimulating pulses. The circuit has a primary power source, a power converter coupled to the primary power source, a signal amplification stage, coupled to the power converter and the signal generator. The signal generator comprises a current mirror, a differential amplifier, a current limiter, and a pair of contacts for non-invasively conveying the pattern of stimulating pulses through the skin of a patient to a subcutaneous nerve. In another embodiment, the circuit may also contain a programming port coupled to a memory for storing a digital waveform.

In a further embodiment, the circuit may be configured to stop generating a signal after a fixed number of uses, a fixed date, an absolute time period, a time period after a first use, a sensed failure, or a feedback signal. In another embodiment, the power converter of the circuit may contain a DC to DC converter.

In still another embodiment the circuit further comprises a first indicator, a second indicator, and a rheostat. The first indicator indicates the mode of the circuit, the second indicator provides an alarm, and the rheostat can be used to adjust the intensity of the stimulating pulses conveyed through the skin of the patient to a subcutaneous nerve. In another embodiment, the first indicator is an LED and the second indicator is an audio speaker or a vibration alert. In still another embodiment, the pattern of stimulating pulses is generated by a lookup table.

In an embodiment, there is disclosed a circuit for non-invasive nerve stimulation comprising a signal microcontroller for storing a digital signal and outputting an analog signal. The signal microcontroller is coupled to a real time clock and a user interface. The user interface may include a switch, an audio generator, and a visual indicator. The circuit also includes a first power supply and a second power supply coupled to the signal microcontroller and the user interface. Where the second power supply contains a voltage converter for increasing the voltage from the first power supply, a signal treatment stage coupled to the signal microcontroller and the second power supply that comprises a current mirror for amplifying and differentiating the analog signal, and at least one conductor connected to the signal treatment stage for non-invasively delivering the analog signal to a patient.

In an embodiment, the circuit of the signal microcontroller is programmed to cease signal generation based on a fault condition, a low voltage condition, a high voltage condition, a date, a programmed period of time, a number of uses, a feedback signal. In still a further embodiment, the circuit's signal microcontroller contains a port.

In another embodiment, a signal generator for generating a signal is disclosed. The signal generator comprises a microprocessor, a primary power source, a power converter coupled to the primary power source, a signal amplification stage, coupled to the power converter, comprising a current mirror, a differential amplifier, and a current limiter, and a pair of contacts for non-invasively conveying the signal to the skin of a patient.

In another embodiment the signal is generated by a look up table. In another embodiment the signal is sampled from an analog signal. In another embodiment the microprocessor is programmed to generate a signal upon activation of the signal generator. In another embodiment In another embodiment the microprocessor comprises a programming port. In another embodiment the microprocessor may be configured to inhibit the signal after at least a fixed number of uses, a fixed date, an absolute time period, a time period after a first use or a sensed failure.

In another embodiment, the circuit contains a feedback detection portion for detecting feedback from the patients skin. In another embodiment, the feedback is a voltage or a current or a temperature.

### INCORPORATION BY REFERENCE

Hereby, all issued patents, published patent applications, and non-patent publications that are mentioned in this specification are herein incorporated by reference in their entirety for all purposes, to the same extent as if each individual issued patent, published patent application, or non-patent publication were specifically and individually indicated to be incorporated by reference.

### BRIEF DESCRIPTION OF THE DRAWINGS

FIG. 1 is a block drawing representing a circuit of the present disclosure.

FIG. 2 is a schematic drawing of an exemplary user interface portion of a circuit of the present disclosure;

FIG. 3 is a schematic drawing of an exemplary signal generation or treatment portion of a circuit of the present disclosure;

FIG. 4 is a schematic drawing of an exemplary secondary power supply portion of a circuit of the present disclosure;

FIG. 5 is a schematic drawing of an exemplary treatment signal output stage portion of a circuit of the present disclosure;

FIG. 6A is a representation of an exemplary output signal of the circuit of the present invention;

FIG. 6B is another representation of an exemplary output signal of the circuit of the present invention;

FIG. 7A is a perspective view of an exemplary device utilizing a circuit board of the present invention;

FIG. 7B is a cut-a-way view of the device of FIG. 7A.

### DETAILED DESCRIPTION

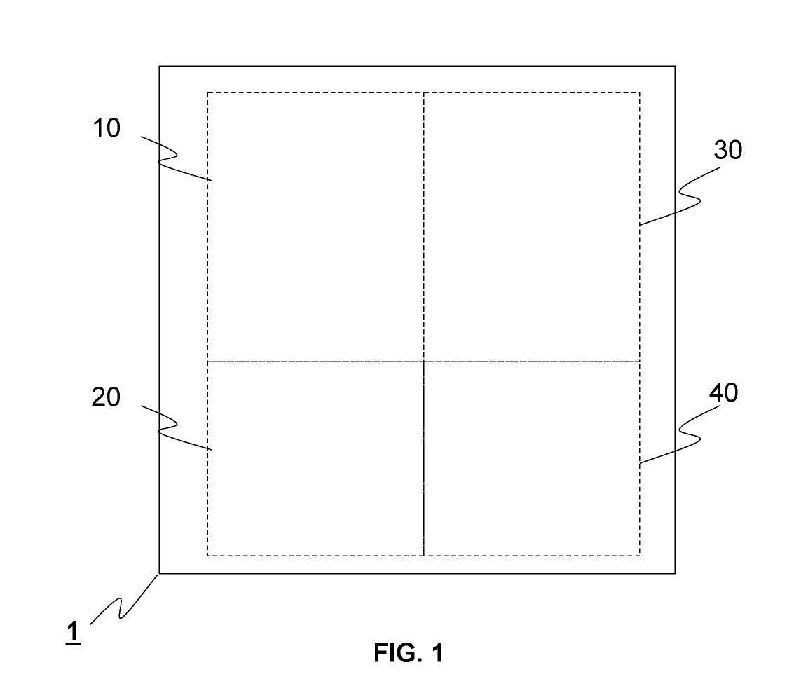

FIG. 1 depicts a block drawing of an exemplary circuit 1 of the present invention. Circuit 1 comprises a user interface portion 10, a signal generation or treatment portion 20, a secondary power supply portion 30 and a treatment signal output stage 40.

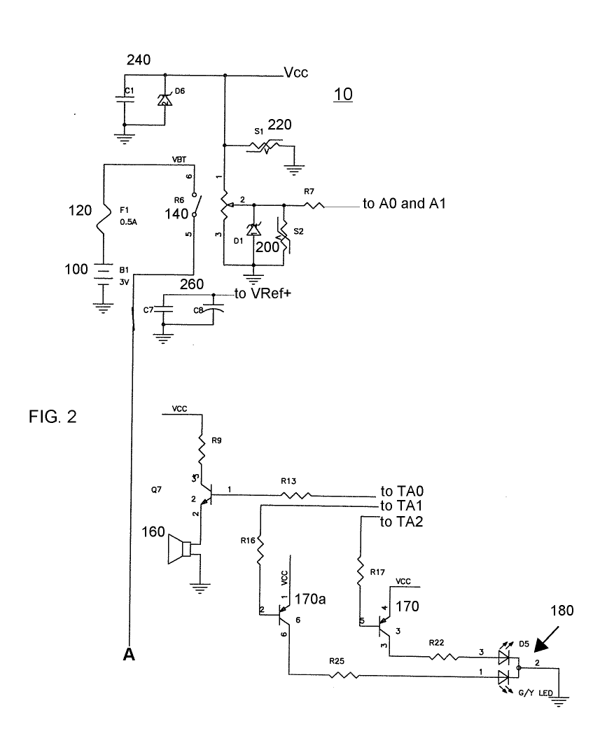

FIG. 2 is a schematic of user interface portion 10. User interface portion 10 comprises DC power source 100, fuse 120, switch 140, audio output 160, LED 180, static protection 200, amplitude adjustment 22, and voltage filters 240 and 260. Power source 100, may be a DC battery or other DC power source such as a DC voltage from an AC transformer. Power source 100 is typically in the 0.1-9 volt range, more typically in the 1.5-3.0 volt range. Power source 100 is connected in series with fuse 120. Fuse 120 is typically a 0.25 to 0.75 A fuse, more typically a 0.5 A fuse, intended to protect the other components from experiencing an over current condition. Fuse 120 may be replaceable if used in conjunction with a fuse holder or may be a single use fuse that is not replaceable. Fuse 120 is connected in series to switch 140. Switch 140 may be a simple on/off type switch or may be a variable rheostat type switch containing a variable resistor R6 to regulate current flow to signal generation or treatment portion 20.

Audio output 160 may be a solid state device that emits audio tones or vibrations indicating the overall status of circuit 1. Audio output 160 is connected in series between the emitter of transistor Q7 and ground. Q7 may be a NPN type surface mount switching transistor, such as an FMMT617. R9 is connected between the collector side of Q7 and Vcc. Vcc is the output of power source 100 through variable resistor R6. The base of Q7 is connected via R13 to an output, TA0 from IC 280 of signal generation section 20. When an audio tone, vibration or other alert is required, output signal TA0 biases transistor Q7, energizing audio output 160.

LED 180 may be a single LED or multiple LEDs. LED 180 may output a single color when energized or may output multiple colors thereby indicating the current state of circuit 10. Typically, LED 180 may be a green/yellow LED and may be used to indicate a power on or standby condition. LED 180 is fed by output signal TA1 or TA2 from pins 23 and 24 of IC 280 respectively. When output signals TA1 or TA2 are enabled, transistors 170 or 170a are biased, respectively, allowing signal Vcc via R16 or R17 to power LED 180 either yellow or green depending on if the device is in the standby state or ready state. Transistors 170 and 170a may be PNP type transistors in a single or dual configuration, such as a BC856S IC containing two high current gain, low collector-emitter saturation voltage transistors in one package.

In an embodiment, the following error table summarizes the possible error codes and the respective LED, audio, and device responses.

TABLE 1

| Errors | Device Reaction |
|---|---|
| System testing | No Beep on Startup |
| Internal Error | Repeated Long Beeps |
| Depleted Battery | No Signal Generated |
| Expired Total Therapy Time | Solid Yellow LED |
| No Treatments Remaining | No Beep |
| No Session Time Remaining | No Signal Generated |
| Low Amount of Total Therapy Time Remains | Flashing Green LED |
| Low Number of Treatments Remaining | No Beep on startup |
| Low Battery | Signal Generated |
| No Errors | 1 Short Beep on Startup |
| | Solid Green LED |
| | No Beep |
| | Signal Generated |

Static protection 200, used to protect signal generation IC 280 from electro static discharge (ESD), may be comprised of a variable resistor 200a and a Zenner diode 200b. Static protection 200 is connected in parallel to the variable resistor side of switch 140 and is used to regulate the analog amplitude input to IC 280. Amplitude adjustment 220 may be a variable resistor with one side tied to Vcc and the other to ground. Varying the resistance may also be used to adjust the analog input signal to IC 280. Voltage filters 240 and 260, used to filter DC signals, may be any well known line type filter configuration, and may be comprised of a number of capacitors and diodes of sufficient parameters to removes transient line voltages.

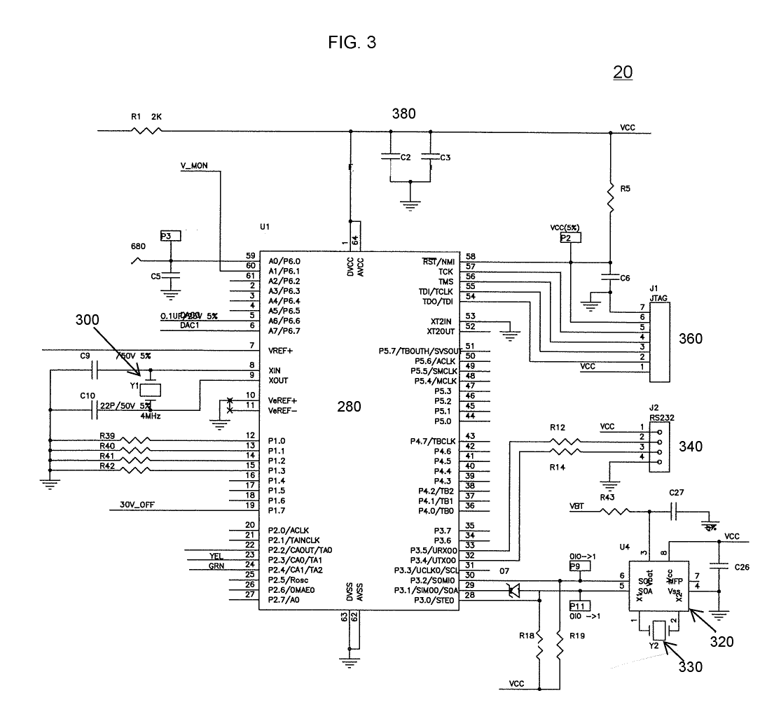

FIG. 3 is a schematic of signal generation portion 20. Signal generation portion 20 comprises signal generator IC 280, oscillator 300, real time clock IC 320, serial port 340, programming port 360 and line filter 380.

Signal generator IC 280 may be a low power microcontroller suitable for operation in a handheld device such as a meter or signal generator. IC 280 must be capable of storing a digital signal file and converting it to an analog output. In an embodiment, a Texas Instruments MSP430F16x ultra low power microcontroller was utilized. The MSP430F16x has five low power modes to optimized extended battery life in portable measurement applications. The IC 280 may comprise a 16-bit RISC CPU, 16-bit registers, and constant generators that contribute to maximum code efficiency. IC 280 comprises a digitally controlled oscillator (DCO) that allows for wake-up from low-power modes to active mode in less than 6 μs. The MSP430F16x series used in an embodiment are microcontroller configurations with two built-in 16-bit timers, a fast 12-bit A/D converter, dual 12-bit D/A converter, one or two universal serial synchronous/asynchronous communication interfaces (USART), I2C, DMA, and 48 I/O pins. In addition, the MSP430F161x series offers extended RAM addressing for memory-intensive applications and large C-stack requirements. Oscillator 300 is connected to IC 280 across input pins Xin and Xout. In an embodiment, a 4 MHz oscillator was utilized, but any oscillator, in the range of about 3 MHz to 5 MHz would work. Connected in parallel with oscillator 300 are capacitors C9 and 10 to filter line noise. C9 and 10 may be 22P/50V 5% capacitors. Contacts 12-15 of IC 280 are all tied to C9 and 10 via resistors R39-42, all of which are then tied to ground.

Real time clock IC 320 is a low-power Real-Time Clocks (RTC) that uses digital timing compensation for an accurate clock/calendar, a programmable output control for versatility, a power sense circuit that automatically switches to the backup supply, and nonvolatile memory for data storage. Using a low-cost crystal, it tracks time using several internal registers. For communication, IC 320 my use the I2C™ bus. The clock/calendar automatically adjusts for months with fewer than 31 days, including corrections for leap years. The clock operates in either the 24-hour or 12-hour format with an AM/PM indicator and settable alarm(s) to the second, minute, hour, day of the week, date or month. Using a programmable signal such as a clock out signal, frequencies of 32.768, 8.192 and 4.096 kHz and 1 Hz can be generated from the external crystal.

Along with the on-board Serial EEPROM and battery backed SRAM memory, a 64-bit protected space is available for a unique ID or MAC address to be programmed at the factory or by the end user. The device may be fully accessible through a serial interface while Vcc is between 1.8V and 5.5V, but can operate down to 1.3V for timekeeping and SRAM retention only. In an embodiment, a MCP794 I2C™ Real-Time Clock/Calendar with EEPROM, SRAM, Unique ID and Battery Switchover from Microchip was utilized. The real time clock IC 320 may be used to keep track of the elapsed time since the unit was first powered on. This may be utilized to preset a lifespan within a device or to signal when the system battery may need to be charged or to monitor the life cycle of the device in which it is installed. Oscillator 330 is connected to real time clock IC 320 via the crystal input and crystal output connections. Oscillator 330 may be a standard 32.768 kHz tuning fork crystal, although other crystals with a comparable specification are also suitable for use as oscillator 330. Vcc powers the IC 320 and the serial clock signal SCL is provided by IC 280 via serial clock line 30. Bidirectional serial data signal SDA is coupled to Vcc via R18 and is tied to the Bidirectional serial data line, 29 of IC 280.

Serial port 340 is a standard 4 pin serial port where pin 1 is tied to Vcc and pin 4 is connected to ground. Pins 3 and 4 may be tied to general purpose I/O ports on IC 280 and are utilized to transmit/receive data to/from IC 280. Serial port 340 may be utilized as a test port or a programming port to set variables, such as number of uses of IC 280 or a lock out date or expiration date.

Programming port 360 is used to program IC 280 with software and a digital wave form profile to be stored in the memory of IC 280. In operation, a digital wave form may be created by sampling an analog signal, generating points on the wave form based on an algorithm, recording points into a table, or utilizing other methods such as mathematical creation, and may be stored in memory of IC 280. In an embodiment, a look up table of signal values was created and stored in memory. In an embodiment, based on the power settings of switch 140, the respective signal values are read from the table and applied to the selected output levels. In operation, regardless of how the signal is created, the digitally stored waveform is converted to an analog signal and output as a differential signal via D/A outputs DAC0 and DAC1.

Pin 1 of port 360 is tied to Vcc and pin 7 is tied to ground and IC 280 reset signal via C6 which may be a 0.1 μf/25v capacitor tied to Vcc. Pins 2-6 are used for inputting software and any other data into IC 280.

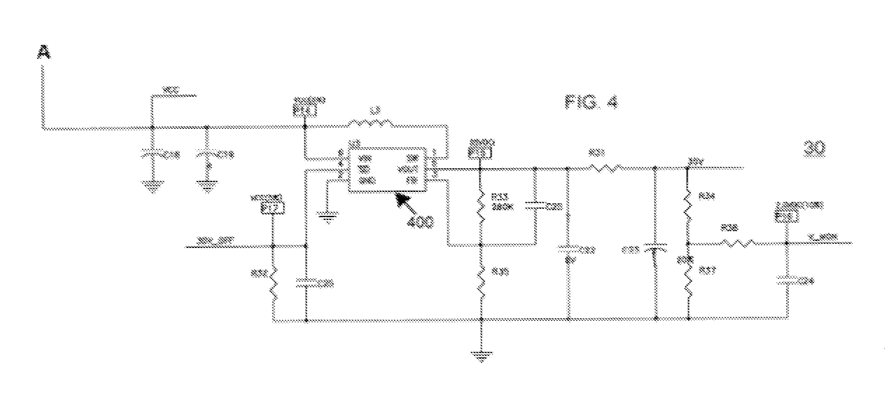

FIG. 4 depicts secondary power supply portion 30. Secondary power supply portion 30 amplifies the input voltage Vcc from power source 100 to generate a final output voltage, typically 35 Volts. IC 400 is a DC to DC converter, such as a LT3461 from Linear Technologies. IC 400 is a general purpose fixed frequency current mode step-up DC/DC converters. It may contain an integrated Schottky and a low VCESAT switch allowing a small converter footprint and reduced parts cost. IC 400 switches at between 1.3 MHz to 3 MHz, thereby enabling the use of very small, low cost and low height capacitors and inductors. The constant switching frequency results in predictable output noise that is easy to filter, and the inductor based topology ensures an input free from switching noise typically present with charge pump solutions. IC 400 contains a high voltage switch rated at 40V making the device ideal for boost converters up to 38V.

Switch pin 1, is connected in series with inductor L3 directly from power supply 100. Pin 3 provides feedback from the output via capacitor C20 and utilizes a reference voltage of approximately 1.255V by connecting a resistor divider tap R33 and R35. Selection of resistors R33 and R35 is in accordance with the desired output voltage from Vout. Vout=1.255V*(1+(R33 value/R35 value). Pin 4 is the shutdown pin to keep secondary power supply portion 30 in a standby mode or in case of error. Pin 4 may be 1.5V or higher to enable the device and approximately 0.4V or less to disable the device. Pin 4 may also aid in functioning as a soft-start for the device by utilizing an RC filter comprised of R32 and C25. Pin 4 is fed by signal 30V_Off from pin 19 of IC 280. Pin 5 is the Vout and is connected to resistor divider R33 and R35. Vout is typically 35 volts in this configuration. Pin 6 is Vin and is tied to the input voltage from power supply 100, directly. Vmon signal 48 is connected through a series of resistors R34 and R38 to Vout and RC network R37 and C24 and is used to feed the monitor voltage back to IC 280 at pin 60.

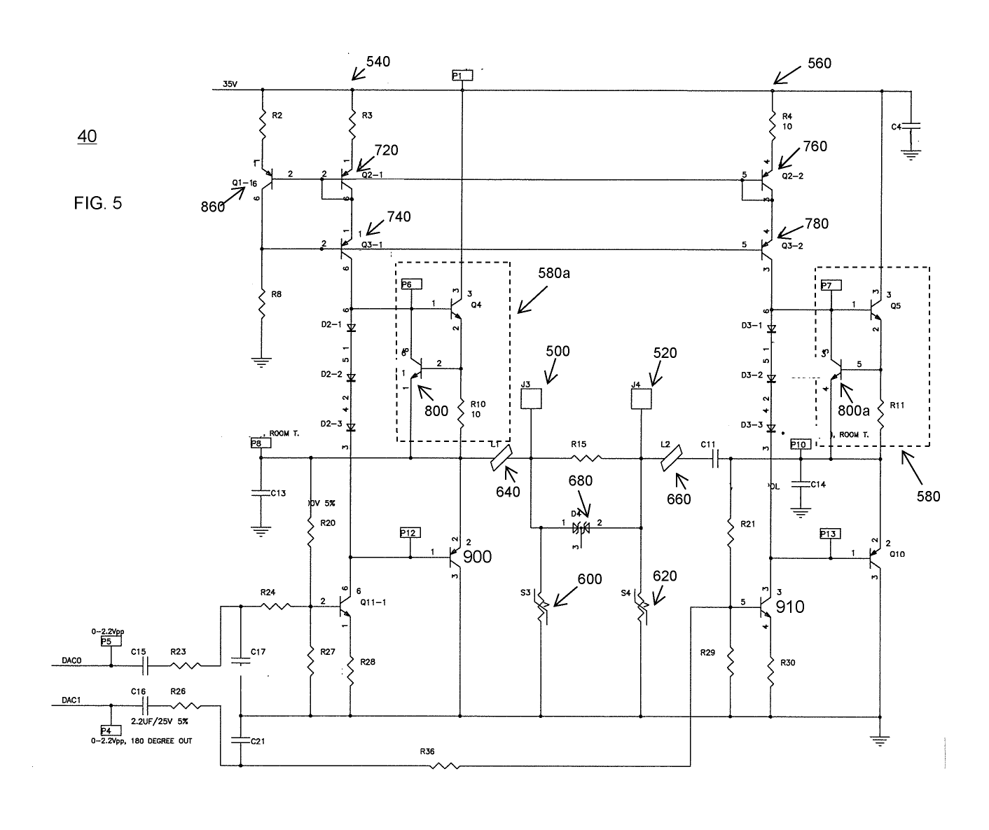

FIG. 5 depicts the treatment signal output stage 40. Treatment signal output stage 40 receives input signals DAC0 and DAC1 from IC 280 and 35 volts from secondary power source 30 and outputs a differential signal at electrodes 500 and 520. Utilizing a voltage clamp with a differential amplifier and current mirrors, output stages 40 modulates and amplifies the output wave for delivery from electrodes 500 and 520. Output stage 40 contains two current mirrors 540 and 560, current limiters 580 and 580a, static protectors 600 and 620, high frequency suppressors 640 and 660, voltage clamp 680, and DC blocking capacitor C11.

Electrodes 500 and 520 do not directly make contact to transmit the signal, but instead may be submerged in conductive gel, for example, SIGNAGEL Electrode Gel from Parker Laboratories, Inc., 286 Eldridge Rd., Fairfield N.J. 07004. Electrodes 500 and 520 may be made of any conductive materials. In the preferred embodiments, electrodes are made of a metal, such as stainless steel. However, in other embodiments, the electrodes may have many other sizes and shapes, and they may be made of other materials [Thierry KELLER and Andreas Kuhn. Electrodes for transcutaneous (surface) electrical stimulation. Journal of Automatic Control, University of Belgrade, 18(2, 2008):35-45; G. M. LYONS, G. E. Leane, M. Clarke-Moloney, J. V. O'Brien, P. A. Grace. An investigation of the effect of electrode size and electrode location on comfort during stimulation of the gastrocnemius muscle. Medical Engineering & Physics 26 (2004) 873-878; Bonnie J. FORRESTER and Jerrold S. Petrofsky. Effect of Electrode Size, Shape, and Placement During Electrical Stimulation. The Journal of Applied Research 4, (2, 2004): 346-354; Gad ALON, Gideon Kantor and Henry S. Ho. Effects of Electrode Size on Basic Excitatory Responses and on Selected Stimulus Parameters. Journal of Orthopaedic and Sports Physical Therapy. 20(1, 1994):29-35.

For example, there may be more than two electrodes; the electrodes may comprise multiple concentric rings; and the electrodes may be disc-shaped or have a non-planar geometry. They may be made of other metals or resistive materials such as silicon-rubber impregnated with carbon that have different conductive properties [Stuart F. COGAN. Neural Stimulation and Recording Electrodes. Annu. Rev. Biomed. Eng. 2008. 10:275-309; Michael F. NOLAN. Conductive differences in electrodes used with transcutaneous electrical nerve stimulation devices. Physical Therapy 71 (1991):746-751]. A more complete description of various embodiments of electrodes can be found in co-pending, commonly-assigned U.S. patent application Ser. No. 13/075,746, filed Mar. 30, 2011, U.S. patent application Ser. No. 13/183,765, filed Jul. 15, 2011, and U.S. patent application Ser. No. 13/222,087 and August 31, the complete disclosures of which are hereby incorporated by reference for all purposes.

Current mirrors 540 and 560 are driven by the 35 volt output of section 30. Transistors 720, 740, 760, 780 and 860 are typical PNP type general purpose transistors and may be single transistors or may be combined in an IC containing several transistors. They should be closely matched with respect to current gain and have low collector-emitter saturation voltage. The current mirrors created by transistor 860 and 720 and the feedback between the base of transistor 720 and its collector allows the current in current mirror 560 to be identical to the current flow in current mirror 540. Similarly, the connection of the bases of transistors 720 and 760 creates the differential signal that inverts DAC0 for output at electrode 500 such that a whole wave form is output.

With the exception of transistor 860 current mirror 560 is the same as current mirror 540. Transistor 860 has its emitter connected to the 35 volts via R2. The collector of transistor 860 is connected to ground via R8, which may be a 180K resistor. The base of transistor 860 is tied to the base and the collector of transistor 720 and the base and collector of transistor 760. Transistors 720 and 760 have their bases shorted to their respective collectors. The emitter of transistor 720 is connected to the 35 volt line through R3. The collector and base of transistor 720 is tied to the emitter of transistor 740. The base of transmitter 740 is coupled to the collector of transistor 860 and the base of transistor 780. The collector of transistor 740 feeds the base of transistor Q4 and may also be connected to a series of diodes. The base of Q4 is tied to the collector of transistor 800 and the emitter of Q4 is tied to the base of transistor 800, creating current limiters 580 and 580a.

The current limiters 580 and 580a, are comprised of a series of Darlington transistors, Q4 and Q5 and 800 and 800a. N-P-N transistors Q4 and Q5 are connected to the 35 volt input on the collector side, and to base of transistors 800, 800a as well as the electrodes 500 and 520 through resistors R10 and R11. Further, transistors Q4 and Q5 are tied, via their base connections to the collectors of transistors 740 and 780. Each current limiter 580 and 580a, clips the output wave at 60 mA and flattens the output wave form delivered to electrodes 500 and 520. Static protectors 600 and 620 may be variable resistors connected between electrodes 500 and 520 and ground. Static protectors high frequency suppressors 640 and 660 may be a series of ferrite beads or any other high frequency suppression method and are connected via DC block capacitor 660 and the emitter side of transistors 800 and 800a. Voltage clamp 680 is connected between electrodes 500 and 520 and may be a pair of Zenner diodes connected cathode to cathode.

Diodes D2 and D3 may provide for shut down voltage protection, i.e., in an over voltage situation, the current will reverse bias the diodes D2 and D2, thereby shutting off the device and preventing injury to the user or damage to the device.

Signals DAC0 and DAC1 from IC 280 are analog outputs of the stored digital signal. DAC0 may be offset from DAC1 by 90 degrees, such that the combined differential signal generated represents a complete sine wave which has been amplified to the proper +/−voltage range, in an embodiment +/−35 volts. Transistor 900 may provide positive amplification of the signal DAC0 while transistor 910 may provide negative amplification of signal DAC1. The 90 degree offset and the combination of the differential signals provides for the complete period of the sine wave.

In operation, power source 100 may be a battery or a series of batteries, and typically outputs 10 volts. Power source 100 is connected to fuse 120 and ground. Switch 140, connected to the other side of fuse 120 may be any type of on off switch, but a variable rheostat type switch that allows the user to adjust the output is preferred. Placing switch 140 into the on position energizes line Vcc. Vcc provides operating voltage and current for ICs 280, 320, and 400, as well as the necessary voltages for LEDs 180, speaker 160, and various other reference voltages.

Once power is applied, secondary power supply 20, amplifies Vcc from power source 100 up to the operational voltage range. In an embodiment, Vcc voltage is about 3 volts and the output of secondary power supply 20 is approximately 35 volts, although other voltage outputs are possible based on the selection of R44 and 46. Voltages in the 20-40 volts range may work, although 35 volts is preferred. Monitor voltage V_mon is sent to IC 280 to monitor the range of output voltages for safety and operational purposes.

IC 280 may be programmed prior to operation utilizing programming port 360. The desired waveform is input in a digital format using port 360 and is stored in a memory portion of IC 280. The input device may be a standard computer, or may be a special signal generator or memory storage device containing the desired wave form. Additionally, IC 280 can be an Application Specific Integrated Circuit (ASIC) with the waveform permanently stored in a memory portion. Alternatively, the wave form may be stored on a separate memory IC device, such as RAM, ROM, or EPROM device. IC 280 may also be programmed with other information such as a counter, timer, use counter, expiration date counter, etc., or any other parameter, utilizing serial port 340. Serial port 340 may also be used as a test port to check for system errors, run diagnostics, read data, etc. Connection with serial port 340 may be accomplished utilizing a standard computer, laptop, tablet, special purpose computer, or any other device capable of reading and writing over a data bus connection.

In an embodiment, the device and or the circuit may detect the level of voltage or current being applied to the skin of the user and may have circuitry or feedback protection sufficient to shut down or limit the out put of the device, to prevent injury to the patient and damage to the device. In an other embodiment, the device and utilize a temperature detector to detect the surface temperature of the conductors of the device and the patient contact points. In an embodiment, based on this feedback, the device may shut down or scale back the applied signal.

Once IC 280 has been programmed, it is ready for operation. Operation occurs, once the system is energized. Based on the programming and signal stored, therein, IC 280 may generate a series of waveforms that are output at DAC0 and DAC1. In one embodiment, an offset sine wave is generated. In other embodiments, square waves, triangular waves, saw tooth waves, etc. may be generated.

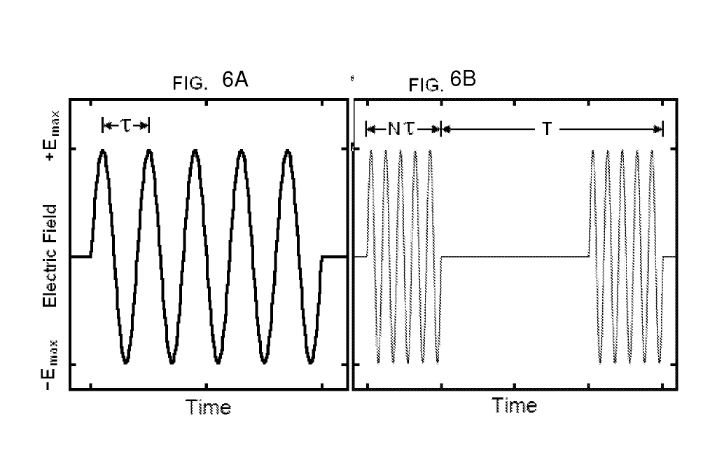

FIGS. 6A and 6B depict a sine wave of an embodiment of the invention. Bursts of sinusoidal pulses are a preferred stimulation waveform, as shown in FIGS. 6A and 6B. As seen there, individual sinusoidal pulses have a period of τ, and a burst consists of N such pulses. This is followed by a period with no signal (the inter-burst period). The pattern of a burst pulse followed by silent inter-burst period repeats itself with a period of T. For example, the sinusoidal period τ may be 200 microseconds; the number of pulses per burst may be N=5, and the whole pattern of burst followed by silent inter-burst period may have a period of T=40000 microseconds (a much smaller value of T is shown in FIG. 6B to make the bursts discernable).

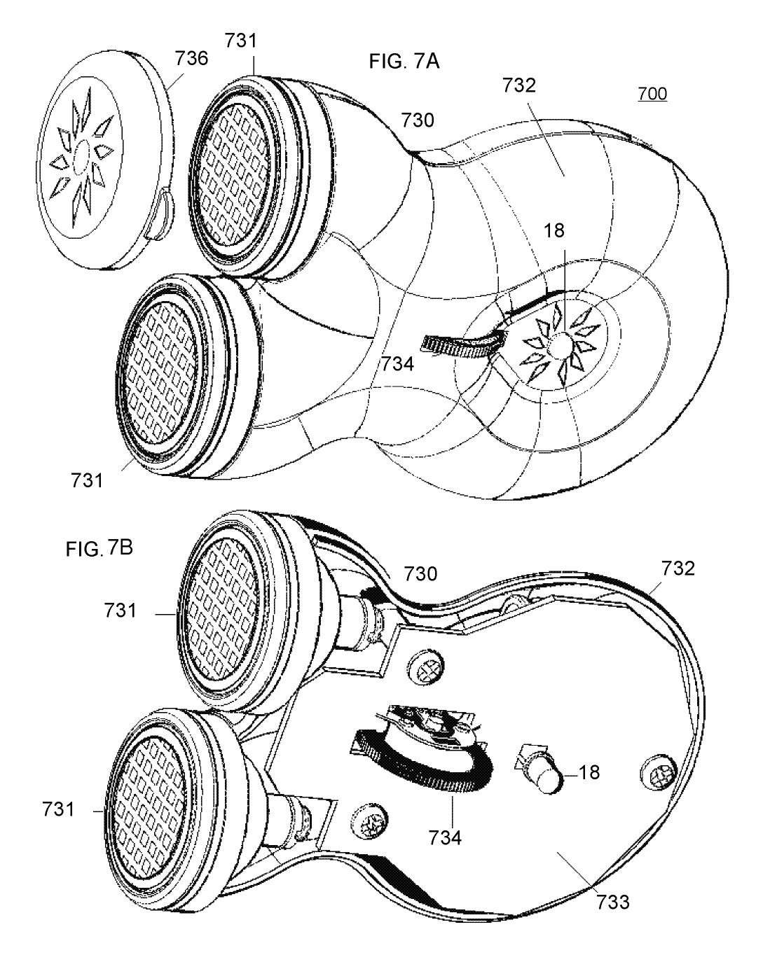

FIGS. 7A and 7B depict an embodiment of a non-invasive nerve stimulator device that utilizes the circuit of the present invention. The circuit depicted in FIG. 1 may be housed inside device, 700, which may also contain power source 100. Alternatively, the circuit of FIG. 1 may be housed in a separate device and coupled to the device depicted in FIG. 7A.

An embodiment of a hand held stimulator is shown in FIG. 7A. A cross-sectional view of the stimulator along its long axis is shown in FIG. 7B. As shown, the stimulator 700 comprises two heads 731 and a body 732 that joins them. Each head 731 contains a stimulating electrode. The body 732 of the stimulator 700 contains the electronic components and battery (not shown) that are used to generate the signals that drive the electrodes, which are located behind the insulating board 733 that is shown in FIG. 7B. However, in other embodiments of the invention, the electronic components that generate the signals that are applied to the electrodes may be separate, but connected to the electrode head 731 using wires. Furthermore, other embodiments of the invention may contain a single such head or more than two heads. Heads of the stimulator 731 are applied to a surface of the patient's body, during which time the stimulator may be held in place by straps or frames (not shown), or the stimulator may be held against the patient's body by hand. In either case, the level of stimulation power may be adjusted with a wheel 734 that may also serve as on/off switch 140. LED 180 is illuminated when power is being supplied to the stimulator. A cap 736 is provided to cover each of the stimulator heads 731, to protect the device when not in use, to avoid accidental stimulation, and to prevent conductive material within the head from leaking or drying. Thus, in this embodiment of the invention, mechanical and electronic components of the stimulator (impulse generator, control unit, and power source) are compact, portable, and simple to operate.

Those skilled in the art will recognize that the present teachings are amenable to a variety of modifications and/or enhancements. For example, although the implementation of various components described above may be embodied in separate hardware devices, it can also be implemented as a single hardware solution—e.g., a custom designed ASIC. While the foregoing has described what are considered to be the exemplary embodiments, it is understood that various modifications may be made therein and that the subject matter disclosed herein may be implemented in various forms and examples, and that the teachings may be applied in other applications, only some of which have been described herein. It is intended by the following claims to claim any and all applications, modifications and variations that fall within the true scope of the present teachings.

1. A circuit for non-invasive nerve stimulator comprising:
a signal generator having an output for generating a pattern of stimulating pulses;
a primary power source;
a power converter coupled to the primary power source;
a signal amplification stage, coupled to the power converter and the signal generator, comprising a current mirror, a differential amplifier, and a current limiter, and
a pair of contacts for non-invasively conveying the pattern of stimulating pulses to the skin of a patient.

2. The circuit of claim 1 further comprising a programming port coupled to a memory for storing a digital waveform.

3. The circuit of claim 1 wherein the circuit may be configured to stop generating a signal after at least one of the following:
a fixed number of uses, a fixed date, an absolute time period, a time period after a first use, a sensed failure and a feedback signal.

4. The circuit of claim 1, wherein the power converter is a DC to DC converter.

5. The circuit of claim 1, further comprising:
a first indicator;
a second indicator; and
a rheostat,
wherein the first indicator indicates the mode of the circuit, the second indicator provides an alarm indication, and the rheostat can be used to adjust the intensity of the stimulating pulses conveyed to the nerve of the patient.

6. The circuit of claim 5 wherein the first indicator is an LED and the second indicator is a audio speaker or a vibrating alert.

7. The circuit of claim 1 comprising a feedback detection portion for detecting feedback from the patient's skin.

8. The circuit of claim 7, wherein the feedback is at least one of the following: a voltage, a current and a temperature.

9. The circuit of claim 1 wherein the pattern of stimulating pulses is generated by a lookup table.

10. A circuit for non-invasive nerve stimulation comprising:
a signal microcontroller for storing a digital signal and outputting an analog signal, the signal microcontroller coupled to a real time clock,
a user interface coupled to the signal microcontroller comprising;
a switch, an audio generator, and a visual indicator;
a first power supply and a second power supply coupled to the signal microcontroller and the user interface,
wherein the second power supply contains a voltage converter for increasing the voltage from the first power supply,
a signal treatment stage, coupled to the signal microcontroller and the second power supply, comprising a current mirror for amplifying and differentiating the analog signal, and at least one conductor connected to the signal treatment stage for non invasively delivering the analog signal to a patient.

11. The circuit of claim 10 wherein the signal microcontroller is programmed to cease signal generation based on at least one of the following parameters:
a fault condition, a low voltage condition, a high voltage condition, a date, a number of uses, and a feedback signal.

12. The circuit of claim 10 wherein the signal microcontroller contains a port.

13. A signal generator for generating a signal comprising:
a microprocessor;
a primary power source;
a power converter coupled to the primary power source;
a signal amplification stage, coupled to the power converter, comprising a current mirror, a differential amplifier, and a current limiter, and
a pair of contacts for non-invasively conveying the signal to the skin of a patient.

14. The signal generator of claim 13 wherein a waveform is generated by the microprocessor using a look up table.

15. The signal generator of claim 13, wherein data points used to generate the signal are sampled from an analog signal.

16. The signal generator of claim 13 wherein the microprocessor is programmed to generate a waveform upon activation of the signal generator.

17. The signal generator of claim 16 wherein a waveform used to generate the signal is generated from a look up table.

18. The signal generator of claim 13 wherein the microprocessor comprises a programming port.

19. The signal generator of claim 13 wherein the microprocessor may be configured to prevent the signal after at least one of the following:
a fixed number of uses, a fixed date, an absolute time period, a time period after a first use, a sensed failure, and receipt of a feedback signal.
